(19) United States
(12) Patent Application Publication
(10) Pub. No.: US 2004/0047846 A1
(43) Pub. Date: Mar. 11, 2004

(54) Ubiquitin promoter in vectors for gene therapy in respiratory tract

(76) Inventor: Stephen Charles Hyde, Oxford (GB)

Correspondence Address: NIXON & VANDERHYE, PC, 1100 N GLEBE ROAD, 8TH FLOOR, ARLINGTON, VA, 22201-4714, US

(21) Appl. No.: 10/296,261

(22) PCT Filed: May 30, 2001
(86) PCT No.: PCT/GB01/02391

(30) Foreign Application Priority Data
May 30, 2000 (GB) 0013108.6

(51) Int. Cl. A61K 48/00; A61L 9/04

(52) U.S. Cl. 424/93.21; 514/44; 424/93.2

(57) ABSTRACT

The human Ubiquitin C promoter is proposed as a highly advantageous promoter for use in airway gene therapy.

### FIELD OF THE INVENTION

The present invention relates to vectors for use in gene therapy, in particular, for example, for directing improved transgene expression for therapeutic purpose in the lung. The vectors of concern include the coding sequence for a therapeutic agent under the control of the human Ubiquitin C (UbC) promoter or a functional analogue of that promoter.

### BACKGROUND TO THE INVENTION

Lung diseases such as cystic fibrosis, asthma, emphysema, pulmonary oedema and lung cancer are suitable for treatment by gene therapy. One of the major limitations, however, of current viral and non-viral based gene therapy vectors for use in airway gene therapy is the short duration of gene expression that can be achieved in the lung. Transgene expression in the lung from current gene therapy vectors which rely on viral promoters typically peaks a few days after dosing and falls away rapidly such that it is undetectable within a few weeks.

Successful gene therapy requires that a therapeutic gene be delivered and expressed in a target cell in vivo at an adequate level and for an adequate time. The field of gene therapy research has concentrated either on the use of (strong) viral promoter elements or (typically weaker) tissue specific promoter elements to direct the desired gene expression. Neither has proven particularly successful in the lung. As indicated above, viral promoter elements, including the widely used immediate early cytomegalovirus (CMV) enhancer/promoter element, are rapidly silenced in the lung. Transcriptional silencing in vivo of viral promoters such as the immediate early CMV promoter appears to be a primitive cellular defence mechanism against viral infection (Yew et al., Human Gene Therapy (1997) 8, 575-584). Lung-specific promoters tend to be extremely weak. Endogenous promoters including those directing expression of human nitric oxide synthase, human mucin 1, rat clara cell 10 kD protein, human ubiquitin B and human interleukin 8 have also shown no enhancement over CMV (Yew et al, Human Gene Therapy 1997, 8: 575-584).

It has previously been shown that the persistence of CMV promoter-mediated transgene expression in mouse lung can be enhanced by the co-expression (either in cis or trans) of the E4 ORF 3 protein derived from serotype 2 adenovirus. However, although persistence of CMV promoter-mediated transgene expression in the lung is enhanced by use of such a vector system, absolute expression levels from day 7 onwards are low (Yew et al., Human Gene Therapy (1999) 10,1833-1843).

With a view to finding improved expression vectors for airway gene therapy which will be of clinical benefit, the inventor has turned to investigation of known promoters of human genes having ubiquitous expression in tissues. Investigation was previously reported of the effectiveness of the Elongation Factor 1a (EF1a) promoter in plasmid DNA for directing expression of the firefly luciferase gene in mouse lung after intranasal administration. For comparison, an identical plasmid was used apart from substitution of the EF1a promoter by the conventionally employed immediate early CMV promoter/enhancer. With the CMV promoter, luciferase expression was maximal after 2 days but essentially undetectable by day 7. While greater persistence of expression of luciferase was observed with the EF1a promoter, reporter gene activity was far lower (7-fold lower at day 2, 38% of day 2 level at day 7 and 23% of day 2 level at day 14; see Abstract 254 of the Proceedings of the 13th Annual North American Cystic Fibrosis Conference, Pediatric Pulmonary Supplement 19, 1994). It has now been found that by substituting the EF1a promoter in the same plasmid vector by the human UbC promoter not only can expression of luciferase in lung comparable to that observed with use of the CMV promoter be achieved but such expression is sustained for a number of weeks. Such expression of a therapeutic agent, e.g. the cystic fibrosis transmembrane conductance regulator gene product in the lungs of cystic fibrosis sufferers, is anticipated to be of clinical benefit.

The human UbC promoter has previously been shown to direct high level recombinant protein expression in a variety of mammalian cell lines (Wulff et al., FEBS Letters (1990) 261, 101-105; Johansen et al., FEBS Letters (1990) 267, 289-294) and in a wide range of tissues of transgenic mice including lung (Shorpp et al., Nucleic Acid Res. (1996) 24, 1787-1788). However, such studies do not enable direct extrapolation as to whether expression vectors relying on the human UbC promoter for expression of a therapeutic agent, when administered to the airways, will provide a sufficient degree and endurance of expression of the desired therapeutic agent for successful gene therapy.

Expression vectors comprising the human UbC promoter have been proposed for delivery of therapeutic genes to the central nervous system (WO 98/32869). However, there has been no suggestion that such vectors may be of use in airway gene therapy. Furthermore, the studies reported in WO 98/32869 do not enable direct extrapolation of the success of expression vectors which rely on the human UbC promoter for expression of a therapeutic agent, when administered to the airways.

The studies reported herein for the first time establish the human UbC promoter as a candidate highly advantageous promoter for gene therapy in the lung.

### SUMMARY OF THE INVENTION

In one aspect, the present invention thus provides the use of a vector including a human Ubiquitin C (UbC) promoter or functional analogue thereof operably-linked to a coding sequence for a therapeutic agent in the manufacture of a medicament for use in airway gene therapy, in a human or non-human animal. As indicated above, such airway gene therapy may particularly be for treatment of cystic fibrosis, asthma, emphysema, pulmonary oedema or lung cancer, especially cystic fibrosis.

In a further aspect, the present invention provides vectors for use in treating cystic fibrosis, emphysema or pulmonary oedema, wherein a human UbC promoter or functional analogue thereof is employed to direct expression of the desired therapeutic agent.

### DETAILED DESCRIPTION

A vector for use in accordance with the invention may be any type of vector conventionally employed for gene therapy. It may be a plasmid expression vector administered as naked DNA or complexed with one or more cationic amphiphiles, e.g. one or more cationic lipids (also called DNA/liposomes, pDNA/liposomes or lipoplex). A viral vector may alternatively be employed, e.g. a recombinant adenovirus such as a recombinant adenovirus of serotype 2,5 or 17, a recombinant adeno-associated virus such as a recombinant adeno-associated virus of serotype 2, a recombinant influenza virus, a recombinant lentivirus such as a recombinant human immunodeficiency virus (HIV), simian immunodeficiency virus (SIV), feline immunodeficiency virus (FIV) or equine infectious anaemia virus (EIAV), or a recombinant retrovirus such as a recombinant moloney murine leukaemia virus or mouse mammary tumour virus.

Thus, for example, a suitable viral vector may be a retroviral vector. Viral vectors may be administered in cells of a retroviral packaging cell line, administered in cell-free form or in the form of producer cells capable of producing the viral vector. Typically, in such a vector, one or more of the genes encoding for the viral proteins (gag, pol and/or env) are replaced by therapeutic and/or marker genes to be transferred to the target cell. Since the replacement of the viral genes leaves the virus unable to replicate, a packaging cell line may be used to produce the viral vector. A packaging cell line will consist of a cell line transfected with one or more plasmids carrying the genes (gag, pol and/or env) enabling the retroviral vector to be packaged. Virtually any cell line can be used to produce a packaging cell line. Preferably, a mammalian cell line is used, for example CV-1, Hela, Raji, SW480, HEK 293 or CHO.

To generate packaged vector, the retroviral vector is transfected into the packaging cell line. The cell line may then be cultured under conditions suitable for the production of packaged retroviral particles by the cell. These particles can readily be obtained from the cells. For example, the cells are cultured and the supernatant harvested. Depending on the desired use, the supernatant containing the particles can be used or these particles can be separated from the supernatant by standard techniques such as gradient centrifugation, filtering etc. The retroviral particles produced in this way may then be used to infect target cells in vitro or in vivo, for example by administration to the airway. A cell infected in this way cannot produce new vector since no viral proteins are present in these cells. However, the DNA of the therapeutic gene is integrated into the cell's DNA and may now be expressed in the infected cell.

A vector may be an integrating gene transfer vector. That is, the vector may have the capacity to integrate into the genome of a cell, such as an airway cell. For example, the vector may be a viral integrating gene transfer vector such as an adeno-associated virus, a retrovirus or a leutivirus. The vector may be a non-viral integrating gene transfer vector such as a synthetic non-viral integrating gene transfer vector, for example a Sleeping Beauty non-viral integrating gene transfer vector.

An expression vector for use in accordance with the invention may include in addition to the human UbC promoter or a functional analogue thereof other control elements conventionally employed in expression vectors operably linked to the coding sequence for the desired therapeutic agent, e.g. a transcription termination sequence and/or a poly A sequence and/or an enhancer element.

The human UbC promoter has previously been cloned and may be obtained, for example, by PCR amplification from the known plasmid pUB6/V5-His A (Invitrogen). By functional analogue of that promoter will be understood any promoter which represents a derivative of the human UbC promoter and retains the ability to sustain expression of the luciferase gene from plasmid DNA in mouse lung in vivo for at least a period of weeks, e.g at least 4 weeks, preferably at least 8 weeks. Preferably such a functional analogue will achieve expression in such an animal model comparable to the maximum obtainable by substitution of the human UbC promoter, e.g at least 50%, more preferably at least 70 to 100% of the maximum expression obtainable with the human UbC promoter. A functional analogue of the human UbC promoter may be an equivalent gene promoter from a non-human mammalian species. It may be a modified human or non-human UbC promoter having one or more base pair substitutions and/or incorporating one or more modified bases.

The therapeutic agent to be expressed will commonly be a protein but may be a nucleic acid or modified nucleic acid. Thus, for example, a vector for use in accordance with the invention to treat cystic fibrosis will include a transgene suitable for substituting for the endogenous cystic fibrosis transmembrane conductance regulator gene. An appropriate cDNA for this may be cloned as previously described or reconstructed from cloned fragments available from the ATCC (see Riordan et al., Identification of the cystic fibrosis gene: cloning and characterization of complementary DNA (1989) 245, 1066-1073; Gil et al., A placebo-controlled study of liposome-mediated gene transfer to the nasal epithelium of pateients with cystic fibrosis, Gene Therapy (1997) 4, 199-209; GenBank Accession no. NM-000492: Human cystic fibrosis transmembrane conductance regulator gene product).

For treatment of emphysema, the human UbC promoter or functional analogue thereof will direct expression of human alpha-1 anti-trypsin or an analogue thereof which is capable of producing a functionally equivalent therapeutic effect. Isolation of the cDNA for human alpha-1 anti-trypsin has also previously been described (see GenBank Accession no. NM-00295 and Ciliberto et al., Cell-specific expression of a transfected human alpha 1-anti-trypsin gene, Cell (1985) 41, 531-540).

As hereinbefore indicated, vectors for use in accordance with the invention are also proposed for treatment of pulmonary oedema In this case, the human UbC promoter or functional analogue thereof may direct expression of the human sodium-potassium-adenosinetriphosphatase enzyme or an analogue of that enzyme which produces the desired therapeutic effect. cDNAs encoding both chains of the human sodium-potassium-adenosinetriphosphatase have also previously been cloned and sequenced (see GenBank accession nos. AH001423 (alpha subunit and U50743 (gamma subunit); see also Sverdlov et al., The family of human Na⁺K⁺-ATPase: No less than five genes and/or pseudogenes related to the alpha-subunit, FEBS Lett. (1987) 217, 275-278).

Various therapeutic agents have previously been proposed for gene therapy treatment of asthma and other chronic inflammatory airway diseases (see, for example, Demoly et al., Gene Therapy (1997) 4, 507-516) and could also be advantageously expressed in the airways by means of an expression vector in accordance with the invention. By way of example of such therapeutic agents, the following are listed: soluble CD40, IL-1R, IL-4R, TNF receptor, IL-10, IL-12, Interferon-γ, TGF-β, and polypeptide inhibitors of the human nuclear factor kappa B transcription factor. cDNAs for such therapeutic agents may be constructed on the basis of protein or gene sequence information or isolated by known methods. See, for example:

GenBank accession no. M27492 (soluble fragment of human IL-R gene product) and Sims et al., Cloning the interleukin 1 receptor from human T cells, Proc. Natl. Acad. Sci USA (1989) 86, 8946-8950;

GenBank accession no.X52425 (soluble fragment of human IL4-R gene product; Idzerda et al., Human interleukin 4 receptor confers biological responsiveness and defines a novel receptor superfamily, J. Exp. Med. (1990) 171, 861-873;

GenBank accession no. U53483(soluble fragment of human TNF receptor gene product; Santee et al., Human tumour necrosis factor receptor p75/80 (CD 120b) gene structure and promoter characterization, J. Biol. Chem. (1996) 271, 21151-21159;

GenBank accession no. X13274 (human IFN-γ gene product); Gray et al., Expression of human immune interferon cDNA in E. coli and monkey cells, Nature (1982) 295, 503-504;

GenBank accession no. M57627 (human IL-10 gene product); Vieira et al., Proc. Natl. Acad. Sci. USA (1991) 88, 1172-1176;

GenBank accession nos. AF180562 and AF180563 (IL-12 chains; p35 and p40 gene products);

GenBank accession no. X02812 (human TGF-β gene product); Derynck et al., Human transforming growth factor-beta complementary DNA sequence and expression in normal and transformed cells, Nature (1985) 316, 701-705.

Vectors for use in accordance with the invention to treat lung cancer may rely on a human UbC promoter or functional analogue thereof to direct expression in the lungs of various therapeutic agents previously proposed for treatment of cancers, including, for example, preferably prodrug-converting enzymes. By prodrug-converting enzyme will be understood a gene product which activates a compound with little or no cytotoxicity into a toxic product. Various prodrug activation strategies employing viral vectors have previously been proposed for cancer treatment (see, for example, Published International Application no. WO 95/07994 and EP-B 0 702 084 of Chiron Corp.) and may be adopted in the lungs by provision of a vector in accordance with the present invention together with the appropriate prodrug. Thus, for example, a vector for use in lung cancer therapy may preferably be constructed such that a human UbC promoter or functional analogue thereof directs expression of a viral thymidine kinase, e.g. Herpes simplex virus thymidine kinase. For prodrug-activation therapy, such an enzyme is employed together with a purine or pyrimidine analogue, e.g. ganciclovir, which is phosphorylated by the viral thymidine kinase to a toxic triphosphate form. Examples of other prodrug-converting enzymes which may be advantageously expressed from a human UbC promoter in the lungs for prodrug activation therapy of lung cancer include:

cytosine deaminase which converts the prodrug 5-fluorocytosine into the toxic compound 5-fluorouracil (Mullen, Proc. Natl. Acad. Sci. USA (1992) 89, 33; see also Efficiacy of adenovirus-mediated CD/5-FC and HSV-1 thymidine kinase/ganciclovir sucide gene therapies concomitant with p53 gene therapy, Xie et al., Clinical-Cancer Res. (1999) 5, 4224-4232);

carboxypeptidase G2 which will cleave the glutamic acid from para-N-bis (2-chloroethyl) aminobenzoyl glutamic acid thereby creating a toxic benzoic acid mustard;

Penicillin-V amidase which will convert phenoxyacetabide derivatives of doxorubicin and melphalan to toxic compounds (Vrudhula et al., J. Med. Chem. (1993) 36, 919-923; Kern et al., Canc. Immun. Immunother. (1990) 31, 202-206);

Platelet-derived endothelial cell growth factor/thymine phosphorylase (PD-ECGF/TP) which converts the prodrug 5′-deoxy-5-fluorouracil (Furtulon) to 5-fluorouracil and 5′-deoxy-D-ribose-1-phosphate (see, for example, Thymidine phosphorylase activity and prodrug effects in a three-dimensional model of angiogenesis; implications for the treatment of ovarian cancer, Stevens et al., Am. J. Pathol. (1998) 153, 1573-1578); and

E. coli nitroreductase which has been utilized with the prodrug CB1954 (The nitroreductase/CB1954 combination in Epstein-Barr virus-positive B-cell lines:induction of bystander killing in vitro and in vivo, Westphal et al., Cancer-Gene-Therapy (January 2000) 7, 97-106).

In a further aspect, the present invention provides a pharmaceutical composition comprising (i) a vector as hereinbefore defined suitable for use in airway gene therapy to treat cystic fibrosis, pulmonary oedema or emphysema and (ii) a pharmaceutically acceptable carrier or diluent. For example, a naked plasmid DNA for use in accordance with the invention may be administered in a physiologically acceptable carrier or diluent, for example, formulation may preferably be as an aqueous preparation. Alternatively, as hereinbefore indicated, an expression vector for use in accordance with the invention may be administered, for example, together with one or more cationic amphiphiles such as one or more cationic lipids, e.g. as a DNA/liposome preparation. Such a preparation may be delivered to the airways, for example, in the form of a water dispersion. Viral vectors for use in accordance with the invention may be formulated in conventional manner for in vivo use, for example in an isotonic physiologic buffer preferably including for example one or more stabilizing additives.

A vector for use in accordance with the invention will generally be administered via the airways, e.g. into the nasal cavity, trachea or lungs, but in some instances intravenous delivery to lung tissue may be permissible and indeed preferred. For example, intravenous delivery of a viral vector in accordance with the invention to treat lung cancer may be preferred where the tumour(s) are readily accessible from the lung capillary bed. Various means of targeting recombinant viral vectors for tissue specific or tumour specific delivery of therapeutic agents have previously been described which may be applied to viral vectors of the invention. Vectors for use in accordance with the invention may be delivered into the airways by, for example, means of a feeding catheter introduced into the nasal cavity or by means of a bronchoscope. Vector delivery for therapy in accordance with the invention may however more preferably be by means of a nebuliser or other aerosolisation device provided the integrity of the vector is maintained.

In a still further aspect, the present invention provides a method of treating an airway- or lung-associated disease, e.g. a disease selected from the group consisting of cystic fibrosis, asthma, pulmonary oedema, emphysema and lung cancer which comprises administering to the airways or lung a vector including a human UbC promoter or functional analogue thereof as hereinbefore described. Suitable dosages for administration of the vector may be determined by appropriate trial. A dosage of 10 to 100 mg DNA may, for example, be found suitable.

The invention is illustrated below with reference to the following examples and figures.

### BRIEF DESCRIPTION OF THE FIGURES

FIG. 1 shows the expression of firefly luciferase in human embryonic kidney 293T cells in vitro, two days after transfection with the plasmids PCIKLux, pUbLux and pEFLux containing the CMV immediate early promoter/enhancer, the human UbC promoter and the human elongation factor 1 alpha promoter respectively, operably-linked to a luciferase coding sequence. Reporter gene expression is given as a percentage of the average reporter gene activity obtained with the CMV promoter.

FIG. 2 shows the expression of firefly luciferase in mouse lung two days after airway administration of the plasmids PCIKLux, pUbLux and pEFLux. Reporter gene expression is given as a percentage of the average reporter gene activity obtained with the CMV promoter.

FIG. 3 shows expression of firefly luciferase in mouse lung following airway administration of the plasmids pCIKLux, pUbLux and pEFLux. Reporter gene expression is given as the percentage of the maximal reporter gene activity obtained with the CMV promoter (day 2 after dosing). The dashed horizontal line in FIG. 1 indicates the reliable sensitivity limit of the assay. The data for each point represents the mean for a group of 5 mice.

FIG. 4 shows the expression of firefly luciferase in mouse lung following airway administration of the plasmid pCIKLux or a Sleeping Beauty (SB) vector plasmid DNA containing the human UbC promoter (SB Ub) or the CMV immediate early promoter/enhancer (SB CMV) operably linked to a luciferase coding sequence. Both integrating and non-integrating SB vectors were used.

### EXAMPLE 1

Comparison of the CMV Immediate Early, Human Elongation Factor 1 Alpha and Human Ubiquitin C Promoters for Directing Protein Expression in Cells Cultured In Vitro.

The effectiveness of the CMV immediate early, human elongation factor 1 alphas and human UbC promoters in directing protein expression in cells grown in culture was compared using a transient plasmid transfection. Plasmid expression vectors were employed containing the human UbC promoter (pUbLux), the CMV immediate early promoter/enhancer (pCIKLux) or the human elongation factor 1 promoter (pEFLux) each directing the expression of a firefly luciferase gene.

Plasmid Construction

The plasmids pUbLux, pCIKLux and pEFLux were constructed starting from the commercially available eukaryotic expression plasmid pCI (Promega, Southampton, U.K.). A PCR fragment containing the human UbC promoter was obtained by PCR amplification from pUB6/V5-His A (Invitrogen, Gronigen, Netherlands). A PCR fragment containing the human Elongation Factor 1 alpha promoter was obtained by PCR amplification from pEF1/V5-His A (Invitrogen, Gronigen, Netherlands).

The plasmid pCI contains the CMV immediate early promoter enhancer positioned 5′ to sequences encoding a hybrid intron (containing the 5′ splice donor site from the first intron of the human β-globin gene and the branch and 3′ splice acceptor site from the intron of an immunoglobulin heavy chain variable region gene), a polylinker for the insertion of a coding sequence to be expressed and the SV40 late polyadenylation signal. Plasmid pCIKLux was constructed by inserting a 1677 bp NheI-NotI restriction fragment (numbering includes the entire restriction enzyme recognition sequences) containing a consensus Kozak translation signal and the firefly luciferase gene from plasmid pKSMKLux into the polylinker of pCI cut with NheI and NotI.

pKSMKLux was constructed by inserting a 1681 bp PCR fragment containing a Kozak translation signal and the firefly luciferase gene amplified from pGL3 (Promega, Southampton, UK) using primers 5′LuxNheKoz and 3′LuxNot into the polylinker of pKSM (Stratagene, Amsterdam. Netherlands) cut with EcoRV.

5′LuxNheKoz 5′-gggctagccaccatggaagacgccaaaaacataaag-3′

3′LuxNot 5′-gggcggccgcctagaattacacggcgatctttccgcc-3′.

A plasmid designated pUb was constructed by replacing the BglII-NheI CMV promoter restriction fragment in pCI with a 1218 bp BglII-NheI restriction fragment (numbering includes the entire restriction enzyme recognition sequences) including the human UbC promoter, exon 1, intron 1 and the 5′ 2 bp of exon 2 (bases −333 to +877 ggcctc . . . ttagac relative to the transcription start site) isolated from plasmid pKSMUb.

pKSMUb was constructed by inserting a 1224 bp PCR fragment containing the UbC promoter (as detailed above) from pUB6/V5-His A using primers 5′BglUb and 3′NheUb into the polylinker of pKSM cut with EcoRV.

5′BglUb 5′-gggagatctggcctccgcgccggg-3′

3′NheUb 5′-ggggctagccgtctaacaaaaaagcc-3′

Plasmid pUbLux was finally constructed by inserting a 1677 bp NheI-NotI restriction fragment (numbering includes the entire restriction enzyme recognition sequences) containing a consensus Kozak translation signal and the firefly luciferase gene from plasmid pKSMKLux into the polylinker of pUb cut with NheI and NotI.

A plasmid designated pEF was constructed by replacing the BgIII-NheI CMV promoter restriction fragment in pCI with a 1324 bp BgIII-NheI restriction fragment (numbering includes the entire restriction enzyme recognition sequences) including the human Elongation Factor 1 alpha promoter, exon 1, intron A, and the 5′ of exon 2 (bases 376 to 1687 of pEF1/V6-His A) isolated from the plasmid pKSMEF.

pKSMEF was constructed by inserting a 1330 bp PCR fragment containing the human Elongation Factor 1 alpha promoter (as detailed above) from pEF1N5-His A using primers 5′EF1A and 3′EF1A into the polylinker of pKSM cut with EcoRV.

5′EF1A 5′-gggagatctgcttttgcaaaaagctttgc-3′

3′EF1A 5′-ggggctagcctatagtgagtcgtattagtacc-3′

Plasmid pEFLux was finally constructed by inserting a 1677 bp NheI-NotI restriction fragment (numbering includes the entire restriction enzyme recognition sequences) containing a consensus Kozak translation signal and the firefly luciferase gene from plasmid pKSMKLux into the polylinker of pEF cut with NheI and NotI.

Gene Transfer

The human embryonic kidney 293T cells were transiently transfected with plasmid DNA in vitro. 2.5×10⁵ 293T cells were plated into 35 mm diameter circular cell culture flasks in 3 ml DMEM cell culture media supplemented with 10% foetal calf serum (Life Technologies, Paisley, UK) and incubated in humidified 5% CO₂ at 37° C. Twenty four hours later, the cell culture media was removed and replaced with 1 μg of the appropriate plasmid DNA, 10 nmol DC0-Chol:DOPE (Gao, X and Huang, L. Biochem Biophys Res Commun 1991; 179:280-285) in 3 ml Opti-MEM 1 (Life Technologies, Paisley, UK) and incubated in humidified 5% CO₂ at 37° C. Four hours later, the plasmid DNA/DC-Chol:DOPE mixture was removed and replaced with 3 ml DMEM cell culture media supplemented with 10% foetal calf serum (Life Technologies, Paisley, UK) and incubated in humidified 5% CO₂ at 37° C.

Assessment of Reporter Gene Expression

The abundance of luciferase reporter gene expression directed by plasmids pCIKLux, pUbLux and pEFLux was assessed in 293T cells cultured in vitro forty eight hours after addition of the plasmid DNA/DC-Chol:DOPE mixture. Cell culture media was removed and a cellular lysate was prepared by resuspending the cells in 300 μl Reporter Lysis Buffer (Promega, Southampton, UK). Reporter gene activity was determined using the Luciferase Assay System of Promega (Southampton, UK). The amount of reporter enzyme activity was determined using standard curves of purified enzyme preparations (Promega, Southampton, UK). Protein concentrations of cellular lysate were determined using a detergent compatible protein assay BioRad, Hemel Hempstead, UK).

Results

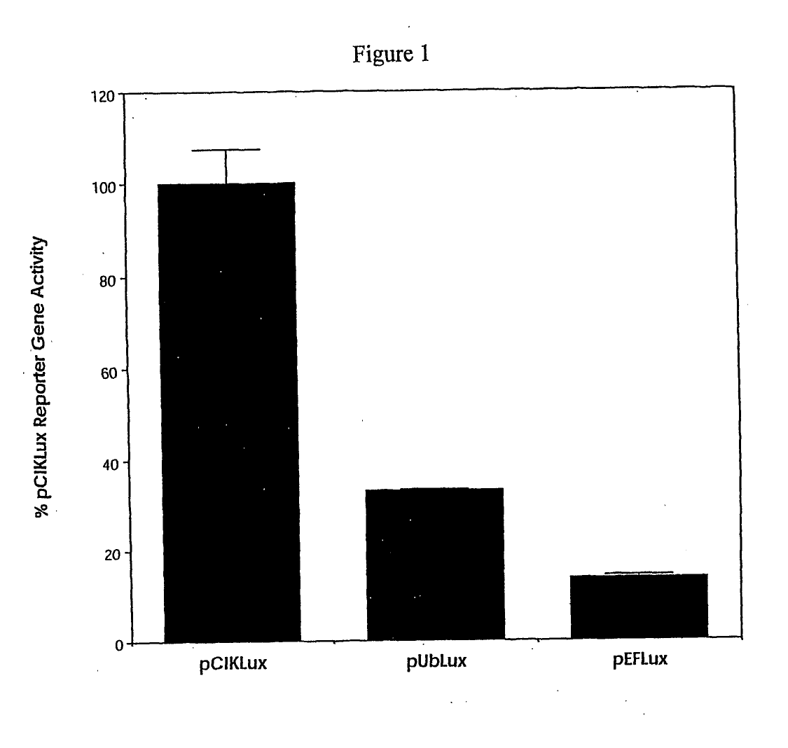

CMV promoter mediated reporter gene expression following transient transfection of 293T cells cultured in vitro was significantly greater than expression directed by either the human UbC or human elongation factor 1 alpha promoters (FIG. 1).

### EXAMPLE 2

Comparison of the CMV Immediate Early Promoters the Human Elongation Factor 1 Alpha Promoter and the Human UbC Promoter for Directing Protein Expression in the Lungs

The effectiveness of the human UbC promoter in directing protein expression in the lungs was studied in a mouse model system employing a plasmid expression vector vector (pUbLux) containing the human UbC promoter directing the expression of a firefly luciferase gene. As a comparison, the same vector was employed but with the human UbC promoter substituted by either the CMV immediate early promoter/enhancer (pCIKLux), or the human Elongation factor 1 alpha promoter (pEFLux). The plasmids were constructed as described in Example 1.

Administration

Plasmid DNA was intranasally instilled into the airways of BALB/c mice (100 μg in 150 μl of water per mouse) after anaethetisation by exposure to the volatile anaesthetic methoxyflurane (Medical Developments Australia Pty Ltd., Springvale, Australia).

Assessment of Reporter Gene Expression

The abundance and persistence of luciferase reporter gene expression directed by pUbLux, pCIKLux and pEFLux was assessed in the lungs and trachea of mice at various time points after plasmid administration up to 182 days (26 weeks). Freshly dissected whole lungs and tracheas were frozen at −80° C. in 200 μl of Reporter Lysis Buffer (Promega, Wisconsin, USA). Tissues were thawed and homogenised for 3×10 seconds using a Ultra-Turrax T8 tissue homogeniser (IKA Labortechnik, Staufen,Germany). Reporter gene activity was assayed using the Luciferase Assay System of Promega (Wisconsin, USA). The amount of reporter enzyme activity was determined using standard curves of purified enzyme preparations (Promega, Wisconsin, USA). Protein concentrations of tissue extracts were determined using a detergent compatible protein assay (BioRad, Hemel Hempstead,UK).

Results

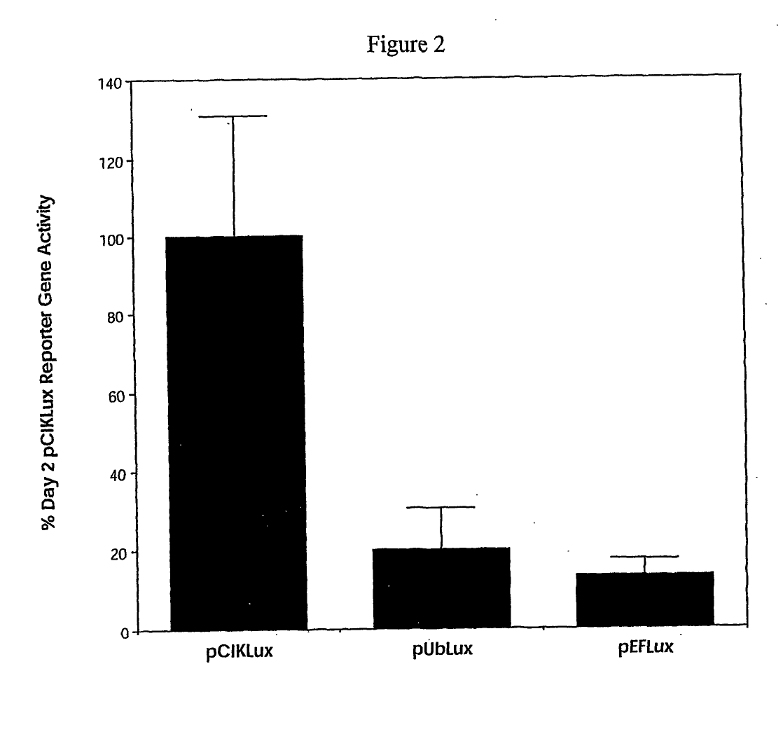

CMV promoter mediated reporter gene expression after 2 days was significantly greater than expression directed by either the human UbC or human elongation factor 1 alpha promoters (FIG. 2).

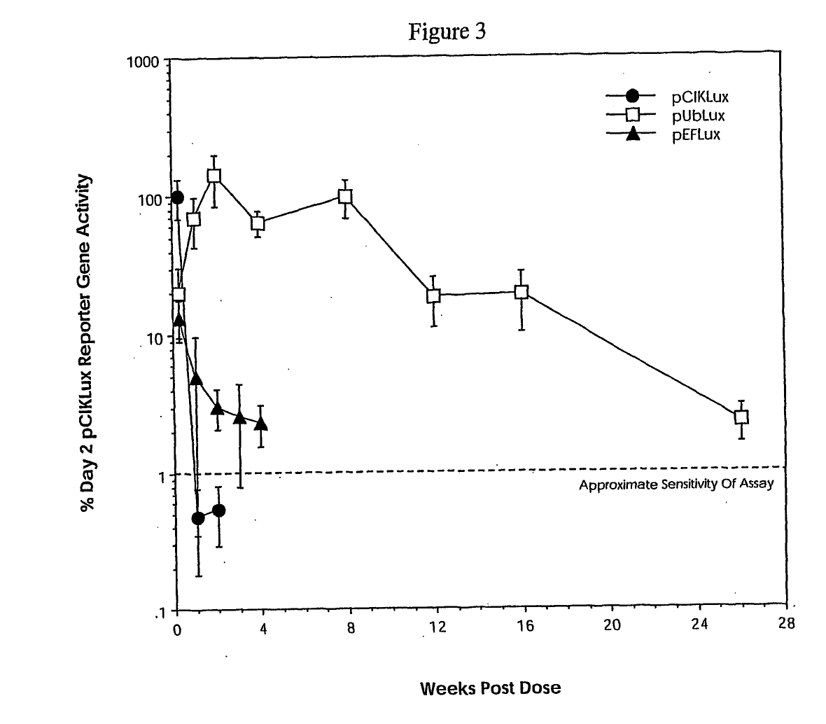

As has been demonstrated by others (Yew et al., Human Gene Therapy (1997) 8, 575-584), CMV promoter mediated lung gene expression was maximal 2 days after dosing and fell to essentially undetectable levels by day 7 (FIG. 3).

The human elongation factor 1 alpha promoter showed some reporter gene expression for up to at least 4 weeks from initial dosing. This expression showed a similar pattern to that seen with the CMV promoter in that the expression values continually fell from their initial day 2 levels (FIG. 3).

In contrast, although the human UbC promoter directed a relatively low level of reporter gene expression at day 2 after dosing, expression of luciferase from pUbLux was found to increase to day 14 and was subsequently sustained at a level similar to the peak expression levels observed with the CMV promoter for up to at least 8 weeks from initial dosing. As shown in FIG. 1, reporter gene expression directed by the human UbC promoter, albeit at a level lower than the maximum obtained with the CMV promoter, was observed even after 26 weeks (182 days) (FIG. 3). Thus, the human UbC promoter directs persistent and abundant reporter gene expression in mouse lung following naked plasmid DNA mediated gene transfer.

### EXAMPLE 3

Comparison of the CMV Immediate Early and Human Ubiquitin C Promoters for Directing Protein Expression in the Lungs Using the Integrating Sleeping Beauty Gene Transfer System.

The effectiveness of the human UbC promoters in directing protein expression in the lungs was studied using a mouse model system employing a Sleeping Beauty non-viral integrating gene transfer vector (Ivics, Z., Hackett, P. B., Plasterk, R. H. and Izsvak, Z. Cell 1997; 91: 501-510) containing the human UbC promoter directing the expression of a firefly luciferase gene (SB Ub). As a comparison, the same vector was employed but with the human UbC promtoer substituted by the CMV immediate early promoter/enhancer (SB CMV).

Gene Transfer Vectors

The plasmids pCMV-SB and pCMV-mSB directing the expression of the Sleeping Beauty transposase and a non functional mutant form of the Sleeping Beauty transposase are described in Ivics, Z., Hackett, P. B., Plasterk, R. H. and Izsvak, Z. Cell 1997; 91: 501-510 and Yant, S. R., Meuse, I., Chiu, W. Ivics, Z., Izsvak, Z and Kay, M. A. Nature Genetics 2000; 25 35-41.

The plasmid pTB/H contains a minimal Sleeping Beauty transposon surrounding a polylinker (Ivics, Z., Hackett, P. B., Plasterk, R. H. and Izsvak, Z. Cell 1997; 91: 501-510).

The plasmids pTB/HUbLux and pTB/HCMVLux were constructed from pTB/H, pCIKLux and pUbLux.

The plasmid pTB/HUbLux was constructed by inserting the 3139 bp BgIII-BamHI restriction fragment (numbering including the entire restriction enzyme recognition sequences) from pUbLux containing the human UbC promoter, exon 1, intron 1 5′ of exon 2, consensus Kozak translation signal, firefly luciferase gene, and SV40 late polyadenylation signal into the unique BgIII site of pTB/H.

The plasmid pTB/HCMVLux was constructed by inserting the 2977 bp BgIII-BamHI restriction fragment (numbering including the entire restriction enzyme recognition sequences) from pCIKLux containing the CMV immediate early promoter/enhancer, consensus Kozak translation signal, firefly luciferase gene, and SV40 late polyadenylation signal into the unique BgIII site of pTB/H.

The SB Ub integrating vector consisted of a 1:9 weight ratio of pCMV-SB and pTB/HubLux respectively.

The SB Ub non-integrating vector consisted of a 1:9 weight ratio of pCMV-mSB and pTB/HUbLux respectively.

The SB CMV integrating vector consisted of a 1:9 weight ratio of pCMV-SB and pTB/HCMVLux respectively.

The SB CMV non-integrating vector consisted of a 1:9 weight ratio of pCMV-mSB and pTB/HCMVLux respectively.

Administration

Sleeping Beauty vector plasmid DNA or pCIKLux plasmid DNA was instilled into the airways of BALB/c mice (100 μg in 150 μl of water per mouse) after anaesthetisation by exposure to the volatile anaesthetic methoxyflurane (Medical Developments Australia Pty Ltd, Springvale, Australia).

Assessment of Reporter Gene Expression

Reproter gene expression after in vivo administration of vectors to mouse lungs was carried out as described in Example 2.

Results

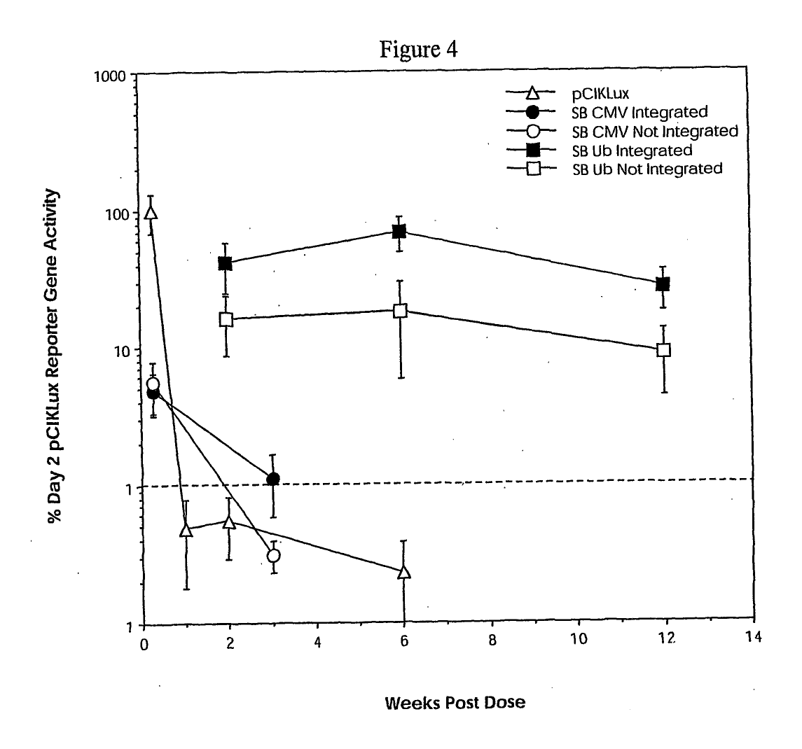

The duration of CMV mediated airway luciferase reporter gene expression was similar if mediated through plasmid DNA (pCIKLux), a non-integrating Sleeping Beauty transposon (SB CMV Non Integrating) or a Sleeping Beauty trasposon with the capacity to integrate into the airway cell genome (SB CMV Integrating). However, as in the context of plasmid DNA (FIG. 3) the duration of human UbC promoter expression was significantly greater than that directed by the CMV immediate early promoter/enhancer in the context of the Sleeping Beauty transposon gene transfer system (FIG. 4). Furthermore, reporter gene expression directed by a non-integrating Sleeping Beauty transposon (SB Ub Non Integrating) was significantly less than a Sleeping Beauty transposon with the capacity to integrate into the airway cell genome (SB Ub Integrating) (p=0.0374 at day 84 post dosing, Mann Whitney Non-Parametric Statistical Analysis).

### EXAMPLE 4

Vector for Use in Cystic Fibrosis Patients

A first vector was made in which the human UbC promoter drives expression of the human cystic fibrosis transmembrane conductance regulator (CFTR) gene (vector pUbCFTR) by replacing the NheI-NotI fragment of pUbLux containing the luciferase coding sequence with the human CFTR cDNA. The NheI-NotI CFTR cDNA fragment was isolated from pCIKCFTR. This plasmid was constructed by inserting a KpnI-NotI fragment containing the entire CFTR cDNA from the plasmid pTRIAL10-CFTR2 into the KpnI-NotI sites in the polylinker of pCI. The construction of plasmid pTRIAL10-CFTR2 has previously been described in Gill et al., Gene Therapy (1997) 4, 199-209.

More preferred analogous vectors for clinical use may be obtained by substituting the ampicillin resistance gene of pUbCFTR by an alternative selectable marker gene, e.g. a kanamycin resistance gene (the FDA preferred plasmid selectable marker for human clinical trials; see FDA document: Points to Consider on Plasmid DNA Vaccines for Preventive Infectious Disease Indications published Dec. 22, 1996 (Docket no. 96N-0400)). Thus, for example, the NheI-NotI fragment of pCIKCFTR containing the human CFTR cDNA may be inserted into plasmid pUbkm which is identical to plasmid pUb except for substitution of the ampicillin resistance gene by a kanamycin resistance gene, to give pUbCFTRkm.

Plasmid pUbCFTR, or more preferably pUbCFTRKm, may be administered to Cystic Fibrosis patients for example either as naked DNA formulated in water or as a plasmid/liposome complex. Delivery may be by instillation into the airways or by means of an aerosol generating device.

### EXAMPLE 5

Vector for Use in Cystic Fibrosis Patients

An alternative plasmid vector for use in Cystic Fibrosis patients can be obtained by inserting the human CFTR gene into the plasmid pVAX (Invitrogen) in which the CMV promoter has also been substituted by the human UbC promoter. Plasmid pVAX is a vector constructed to be consistent with the above-noted FDA document and contains in addtion to a CMV promoter, the origin of replication from plasmid pMB1 and a kanamycin resistance gene.

1. Use of a vector including a human Ubiquitin C (UbC) promoter or functional analogue thereof operably-linked to a coding sequence for a therapeutic agent in the manufacture of a medicament for use in airway gene therapy.

2. Use of a vector according to claim 1 wherein the human UbC promoter is operably-linked to a protein coding sequence.

3. Use of a vector according to claim 1 or 2 which is a plasmid vector.

4. Use of a vector according to claim 1 or 2 which is a viral vector.

5. Use of a vector according to any one of claims 1 to 4 which is an integrating gene transfer vector.

6. Use of a vector according to claim 5 which is a Sleeping Beauty vector.

7. Use of a vector according to any one of claims 1 to 6 wherein said airway gene therapy is for treatment of cystic fibrosis, asthma, emphysema, pulmonary oedema or lung cancer.

8. A vector as defined in any one of claims 1 to 6 for use in treating cystic fibrosis.

9. A vector according to claim 8 which comprises a cystic fibrosis transmembrane conductance regulator gene.

10. A vector as defined in any one of claims 1 to 6 for use in treating emphysema.

11. A vector as defined in any one of claims 1 to 6 for use in treating pulmonary oedema.

12. A pharmaceutical composition comprising a vector according to any one of claims 8 to 11 together with a pharmaceutically acceptable carrier or diluent.

13. A method of treating an airway- or lung-associated disease which comprises administering to the airways or lung a vector as defined in any one of claims 1, 4 or 5.

14. A method as claimed in claim 13 wherein said disease is selected from the group consisting of cystic fibrosis, asthma, emphysema, pulmonary oedema and lung cancer.
